(19) United States
(12) Patent Application Publication
(10) Pub. No.: US 2014/0121716 A1
(43) Pub. Date: May 1, 2014

(54) HIGH VOLTAGE THERAPY DIVERSION ALGORITHMS

(71) Applicant: MEDTRONIC, INC., Minneapolis, MN (US)

(72) Inventors: David A. Casavant, Reading, MA (US); Mark E. Gibbs, Granby, MA (US); Bruce D. Gunderson, Plymouth, MN (US); Robert A. Betzold, Fridley, MN (US)

(73) Assignee: Medtronic, Inc., Minneapolis, MN (US)

(21) Appl. No.: 13/665,183

(22) Filed: Oct. 31, 2012

(51) Int. Cl. A61N 1/39; A61N 1/365

(52) U.S. Cl. 607/4; 607/5

(57) ABSTRACT

An implantable medical device capable of delivering high voltage therapy includes a therapy delivery module comprising a high voltage therapy delivery circuit, a high voltage short circuit protection circuit configured to terminate delivery of a high voltage pulse by the therapy delivery module in response to a short circuit condition, and a sensing module for detecting a need for a high voltage therapy. The device further includes a therapy control unit configured to control the therapy delivery module to deliver a shock pulse in response to detecting the need for the high voltage therapy. The control unit detects a termination of the high voltage pulse by the protection circuit; a truncated shock charge remaining on the high voltage therapy delivery circuit upon terminating the high voltage pulse. The control unit controls the therapy delivery module to deliver a next shock pulse at the remaining truncated shock charge.

### FIELD OF THE DISCLOSURE

The disclosure relates generally to medical devices configured to deliver a high voltage therapy. In particular the disclosure relates to devices and methods for diverting a high voltage therapy in response to a high voltage short circuit condition.

### BACKGROUND

Implantable cardioverter defibrillators (ICDs) typically have the capability of delivering both low voltage therapies and high voltage therapies in response to monitoring a cardiac rhythm and detecting a need for therapy. Low voltage therapies may include bradycardia pacing, cardiac resynchronization therapy (CRT), and anti-tachycardia pacing (ATP). Low voltage therapies are typically delivered using low voltage pacing electrodes, e.g. tip or ring electrodes delivering pulses of 5 Volts or less in amplitude. High voltage therapies such as cardioversion or defibrillation shocks are delivered in response to detecting ventricular tachycardia or ventricular fibrillation. High voltage therapies are typically delivered using high voltage coil electrodes and the housing of the ICD, often referred to as the “CAN electrode” or a “housing electrode.” High voltage electrodes generally have a greater surface area and deliver high energy shock pulses, typically in the range of at least 10 Joules and up to 35 Joules. A single lead may carry multiple electrodes, which may include either or both high voltage and low voltage electrodes. Each electrode is coupled to an electrically insulated conductor extending through the elongated lead body to facilitate electrical connection of each therapy delivery electrode to the ICD.

Short circuit conditions can sometimes occur when a therapy delivery electrode or its conductor makes electrical contact with another conductor or electrode. Lead integrity testing may be performed regularly to make lead measurements, such as lead impedance measurements, to monitor for possible short circuit or other lead conditions. Low voltage short circuit conditions can be readily detected using such measurements. However, a non-contact high voltage lead fault can exist and manifest only when a high-voltage therapy is delivered, causing arcing between exposed conductors. These types of faults involving high voltage conductors are frequently undetected by low voltage lead measurements. A high voltage short circuit that occurs during delivery of a defibrillation shock is likely to prevent adequate energy from being delivered to the heart, leading to a failed therapy. Since ventricular fibrillation is a life-threatening condition, prompt detection of a high voltage short circuit condition, appropriate circuit protection and diversion of a high voltage therapy is needed to provide the possibility of successfully delivering a therapy to a patient.

### SUMMARY

An implantable medical device (IMD) capable of delivering high voltage therapy detects a high voltage short circuit condition, protects the device circuitry from the high voltage short circuit, and responds to the high voltage short circuit condition by controlling a therapy delivery unit to deliver therapy in an altered manner. The IMD includes a therapy delivery module comprising a high voltage therapy delivery circuit, a high voltage short circuit protection circuit configured to terminate delivery of a high voltage pulse by the therapy delivery module in response to a short circuit condition, and a sensing module for detecting a need for a high voltage therapy. The device further includes a therapy control unit configured to control the therapy delivery module to deliver a shock pulse in response to detecting the need for the high voltage therapy. The control unit detects a termination of the high voltage pulse by the protection circuit resulting in a truncated shock charge remaining on the high voltage therapy delivery circuit upon terminating the high voltage pulse. The control unit controls the therapy delivery module to deliver a next shock pulse at the remaining truncated shock charge, without adjustment of the capacitor charge prior to delivering the remaining truncated shock charge. In various embodiments, the control unit may select an alternate electrode vector, alternate electrode polarity or a combination of pacing electrodes for delivering a truncated shock charge. The control unit may be configured to deliver anti-tachycardia pacing therapy subsequent to detecting a terminated shock pulse. The control unit may be configured to deliver high voltage stimulation pulses for activating the diaphragm subsequent to detecting a terminated shock pulse. Other aspects and embodiments of the IMD and associated methods of use will be described.

### BRIEF DESCRIPTION OF THE DRAWINGS

FIG. 1 is a schematic representation of an implantable medical device (IMD) capable of delivering high voltage and low voltage therapies to a heart.

FIG. 2 is a functional block diagram of the IMD shown in FIG. 1 according to an illustrative embodiment.

FIG. 3 is a flow chart of a method for controlling delivery of a HV shock therapy to a patient.

FIG. 4 is a flow chart of a method for controlling electrical stimulation therapy in response to detecting a shockable rhythm according to an alternative embodiment.

### DETAILED DESCRIPTION

In the following description, references are made to illustrative embodiments. It is understood that other embodiments may be utilized without departing from the scope of the disclosure. As used herein, the term “module” refers to an application specific integrated circuit (ASIC), an electronic circuit, a processor (shared, dedicated, or group) and memory that execute one or more software or firmware programs, a combinational logic circuit, or other suitable components that provide the described functionality.

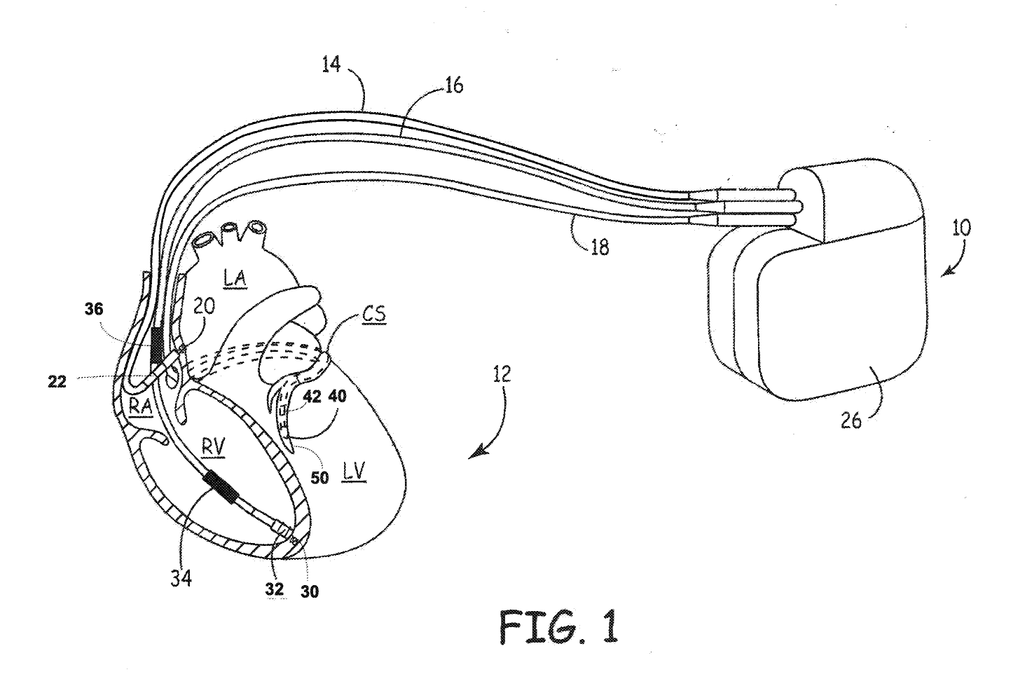

FIG. 1 is a schematic representation of an implantable medical device (IMD) 10 capable of delivering high voltage and low voltage therapies to heart 12. IMD 10 is coupled to heart 12 via leads 14, 16 and 18. Right atrial lead 14 extends from IMD 10 to the right atrium (RA) and carries distal electrodes 20 and 22 for sensing cardiac electrical signals and delivering pacing pulses in the RA.

Right ventricular lead 16 carries a tip electrode 30 and a ring electrode 32 for sensing cardiac electrical signals and delivering pacing pulses in the RV. RV lead 16 additionally carries high voltage coil electrodes 34 and 36, referred to herein as the RV coil electrode 34 and the superior vena cava (SVC) coil electrode 36, for delivering high voltage cardioversion and defibrillation shocks in response to detecting a shockable tachyarrhythmia from sensed cardiac signals. In addition, a housing electrode 26, also referred to as a CAN electrode, can be formed as part of the outer surface of the housing of IMD 10 and be used as an active electrode in combination with coil electrodes 34 and/or 36 during shock delivery.

A coronary sinus (CS) lead 18 is shown extending into a cardiac vein 50 via the RA and coronary sinus for positioning electrodes 40 and 42 for sensing cardiac signals and delivering pacing pulses along the left ventricle. In some examples, CS lead 18 may additionally carry electrodes for positioning along the left atrium for sensing and stimulation along the left atrial chamber.

The depicted positions in or about the right and left heart chambers are merely illustrative. Other leads and pace/sense electrodes and/or high voltage electrodes can be used instead of, or in combination with, any one or more of the depicted leads and electrodes shown in FIG. 1. Lead and electrode configurations are not limited to transvenous leads and intravenous or intracardiac electrodes as shown in FIG. 1. In some embodiments, an IMD system may include subcutaneous electrodes, which may be carried by an extravenous lead extending from IMD 10 or leadless electrodes incorporated along the IMD housing.

IMD 10 is shown as a multi-chamber device capable of sensing and stimulation in three or all four heart chambers. It is understood that IMD 10 may be modified to operate as a single chamber device, e.g. with a lead positioned in the RV only, or a dual chamber device, e.g. with a lead positioned in the RA and a lead positioned in the RV. In general, IMD 10 may be embodied as any single, dual or multi-chamber device including lead and electrode systems for delivering at least a high voltage therapy and may be configured for delivering both high voltage shock pulses and low voltage pacing pulses.

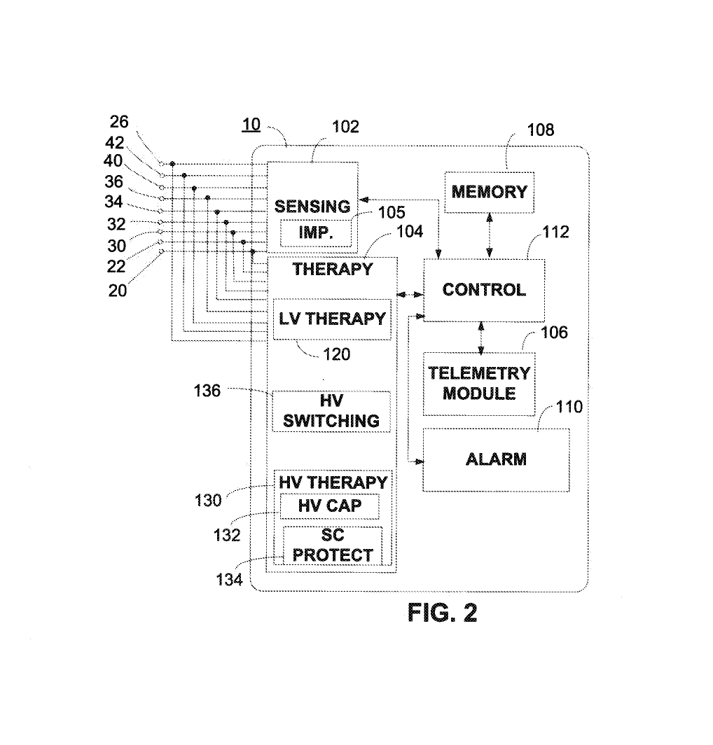

FIG. 2 is a functional block diagram of the IMD 10 shown in FIG. 1 according to an illustrative embodiment. IMD 10 includes a sensing module 102, a therapy delivery module 104, a telemetry module 106, memory 108, and a control unit 112, also referred to herein as “controller” 112.

Sensing module 102 is coupled to electrodes 20, 22, 30, 32, 34, 36, 40, 42 and housing electrode 26 (all shown in FIG. 1) for sensing cardiac electrogram (EGM) signals. Sensing module 102 monitors cardiac electrical signals for sensing signals attendant to the depolarization of myocardial tissue, e.g. P-waves and R-waves, from selected ones of electrodes 20, 22, 26, 30, 32, 34, 36, 40, and 42 in order to monitor electrical activity of heart 12. Sensing module 102 may include a switch module to select which of the available electrodes are used to sense the cardiac electrical activity. The switch module may include a switch array, switch matrix, multiplexer, or any other type of switching device suitable to selectively couple electrodes to sensing module 102. In some examples, controller 112 selects the electrodes to function as sense electrodes, or the sensing vector, via the switch module within sensing module 102.

Sensing module 102 may include multiple sensing channels, each of which may be selectively coupled to respective combinations of electrodes 20, 22, 26, 30, 32, 34, 36, 40, and 42 to detect electrical activity of a particular chamber of heart 12, e.g. an atrial sensing channel and a ventricular sensing channel. Each sensing channel may comprise an amplifier that outputs an indication to controller 112 in response to sensing of a cardiac depolarization, in the respective chamber of heart 12. In this manner, controller 112 may receive sense event signals corresponding to the occurrence of R-waves and P-waves in the various chambers of heart 12. Sensing module 102 may further include digital signal processing circuitry for providing controller 112 with digitized EGM signals, which may be used to measure EGM signal features or for signal morphology analysis in some embodiments.

Sensing module 102 and control unit 112 are configured to monitor the patient's cardiac rhythm for determining a need for therapy delivery and for timing therapy delivery. In response to detecting a tachyarrhythmia, controller 112 controls therapy delivery module 104 to deliver a therapy according to programmed therapies stored in memory 108.

Sensing module 102 may include impedance monitoring circuitry 105 for measuring current between a measurement pair of electrodes 20 through 42 in response to a drive signal. The drive signal is generally a low voltage signal, and impedance measurements may be used by control 112 to detect low voltage short circuit conditions or other lead-related issues detectable when a low voltage drive signal is used. Such low voltage impedance measurements may be performed periodically or in response to loss of pacing capture or a change in pacing threshold to detect lead-related issues.

In some embodiments, impedance is measured during delivery of a high voltage therapeutic shock to detect a HV short circuit condition. When a HV shock is delivered, a breach in the insulation of a lead conductor is detected as a result of arcing (i.e. capacitive coupling) that occurs between highly charged electrical conductors of opposite electrical polarity within a lead body, or between a conductor having a compromised conductor and the electrically active IMD can electrode 26. This type of HV short circuit condition is not typically detected during routine LV impedance measurements. Routine testing using high voltage shocks are impractical as high voltage shocks result in significant patient discomfort. Impedance monitoring during delivery of a HV shock therapy may be used, however, to detect a HV short circuit condition, enabling controller 112 to respond to the HV short circuit condition as will be described in greater detail below. Accordingly, in one embodiment, an impedance monitoring circuit 105 provides controller 112 or short circuit (SC) protection circuit 134 an impedance signal during a high voltage shock delivery to enable termination of the shock and detection of shock termination by controller 112.

Therapy delivery module 104 is coupled to electrodes 20, 22, 26, 30, 32, 34, 36, 40, and 42 for delivering electrical stimulation therapy to the patient's heart. In some embodiments, therapy delivery module 104 includes low voltage (LV) therapy circuitry 120 including a pulse generator for generating and delivering LV pacing pulses during bradycardia pacing, cardiac resynchronization therapy (CRT), and anti-tachycardia pacing (ATP). Control unit 112 controls LV therapy circuitry 120 to deliver pacing pulses according to programmed control parameters using electrodes pacing electrodes 20, 22, 30, 32, 40 and/or 42 for example. Electrodes 20, 22, 30 32, 40 and 42 are generally referred to a “low voltage” electrodes because they are normally used for delivering relatively low voltage therapies such as pacing therapies as compared to the high voltage therapies, i.e. cardioversion and defibrillation therapies, delivered by high voltage coil electrodes 32 and 34. However, as will be described herein, in some instances LV electrodes 20, 22, 30, 32 40 and 42 may be used for delivering a high voltage therapy in response to detection of a high voltage short circuit condition.

Therapy delivery module 104 includes high voltage (HV) therapy delivery circuitry 130 for generating and delivering high voltage cardioversion and defibrillation shock pulses. HV therapy delivery circuitry 130 includes HV capacitors 132 that are charged in response to detecting a shockable cardiac rhythm, e.g. a ventricular tachycardia or ventricular fibrillation. After determining HV capacitors 132 have reached a targeted charge voltage, according to a programmed shock energy, HV therapy delivery 130 delivers a shock pulse via selected HV electrodes, e.g. coil electrodes 34, 36 and housing electrode 26.

HV therapy circuitry 130 includes short circuit (SC) protection circuitry for protecting IMD 10 against a short circuit fault during HV therapy delivery. In one embodiment, SC protection circuitry 134 monitors the current during the shock pulse delivery and in response to a relatively high current, i.e. very low impedance, SC protection circuitry 134 immediately terminates the shock pulse, e.g. by an electronic switch, to prevent damage to the circuitry of IMD 10. The HV short circuit condition would prevent delivery of the HV shock to the heart and would fail to terminate a detected shockable rhythm. By protecting the IMD circuitry from the SC fault, controller 112 remains operable to alter the HV therapy delivery to still treat the tachyarrhythmia and/or control therapy delivery module 104 to deliver alternative electrical stimulation therapies. Accordingly, in one embodiment, controller 112 receives a signal from SC protection circuit 134 indicating a short circuit condition is present and a shock has been terminated. For example a circuit breaker may be included in SC protection circuitry 134 which may open in response to a higher than expected current flow during HV shock delivery. A signal may be received by controller 112 indicating the SC protection circuitry has been activated to cause termination of the HV shock pulse.

In response to receiving a HV short circuit condition signal, e.g. from SC protection circuitry 134 or impedance measuring circuitry 105, controller 112 may store in memory 108 an electrode vector and polarity combination being used to deliver the HV shock pulse that resulted in the short circuit condition. This information may be retrieved and used by a clinician in resolving the HV short circuit condition, e.g. by replacing a lead or reprogramming the therapy delivery electrode configuration and polarity. This information may be used by controller 112 in selecting electrode vectors and polarities for delivering future HV therapies, including an electrode combination and polarity assignment used for delivering a truncated shock charge after premature termination of a shock pulse by SC protection circuitry 134, as will be described below.

Therapy delivery module 104 includes HV switching circuitry 136 used for controlling the pathway through which HV capacitors 132 are discharged. HV switching circuitry 136 may include a switch array, switch matrix, multiplexer, or any other type of switching device suitable to selectively couple combinations of low voltage electrodes (e.g. electrodes 20, 22, 30, 32, 40 and 42) and/or high voltage electrodes (e.g. electrodes 34 and 36) and housing electrode 26 to HV therapy circuitry 130. In some examples, controller 112 selects a shock vector using any of HV coil electrodes 34, 36 and housing electrode 26. As will be described below, controller 112 may select the polarity of the electrodes included in the shock vector using switching circuitry 136.

In some embodiments, the HV capacitors may be coupled to multiple pacing electrode cathodes simultaneously, e.g. any combination or all of LV electrodes 20, 22, 30, 32, 40 and 42 for delivering a HV shock in response to a HV short circuit condition. The anode may be any of the coil electrodes 34, 36, housing electrode 26 or combination of remaining LV electrodes 20, 22, 30, 32, 40 and 42 or any other housing based or lead based electrodes that may be available in the particular IMD system. Pacing capacitors coupled to electrodes 20, 22, 30, 32, 40 and 42 included in LV therapy circuitry 120 may be used in distributing the HV charge remaining on the HV capacitor(s) 132 in some embodiments in an attempt to deliver a needed shock therapy. In this case the pacing capacitors are rated for adequately high voltage to distribute the shock energy among selected electrodes.

The controller 112 may control HV switching circuitry 136 to deliver high voltage pacing pulses using any combination of electrodes 20, 22, 30, 32, 40 and 42 for achieving diaphragmatic muscle activation in response to detecting a HV short circuit condition. Strong diaphragm contractions are induced to provide a cardiac resuscitative effect, which may have an effect similar to external chest compressions, by cyclically increasing and decreasing pressure within the thoracic cavity by changing the volume of the thoracic cavity as the diaphragm contracts and relaxes. This cyclic pressure change of the thoracic cavity may increase cardiac output of the tachyarrhythmic heart.

Diaphragmatic activation may occur via left and/or right phrenic nerve stimulation or via direct stimulation of the diaphragm. It is expected that left phrenic nerve stimulation will occur via electrodes 40 and 42 situated along the left ventricle, and right phrenic nerve stimulation will occur via electrodes 20 and 22 situated in the right atrium though any electrodes available may be used to achieve diaphragm activation.

High voltage pacing pulses used to achieve diaphragmatic activation will have a greater voltage than typical cardiac pacing pulses and will generally have a pulse energy intermediate cardiac pacing pulses and cardiac shock pulses. In order to achieve diaphragmatic activation to cyclically decrease and increase pressure in the thoracic cavity, electrical pulses may be delivered having an amplitude up to a maximum of approximately 100 Volts. Pulses for activating the diaphragm may be delivered at a rate of between approximately 40 and 80 pulses per minute, or for example at a rate of approximately 60 to 70 pulses per minute to achieve a cyclical thoracic cavity pressure change to increase cardiac output. In one embodiment, “approximately” refers to a value that is within 10% of a stated value. For example, diaphragmatic pacing pulses may be delivered between 10 V±10% and 100 V±10%.

The high voltage diaphragmatic pacing pulses may be delivered by discharging HV capacitor 132 via HV switching circuitry 136 to selected pacing electrodes 20, 22, 30, 32, 40 and 42, which may include using high-voltage rated pacing capacitors typically used for delivering HV pulses, which may be implemented in either LV therapy delivery circuitry 120 or in HV therapy delivery circuitry 130, for distributing the pacing energy to the selected electrodes. For example, electrical pacing pulses for activating the diaphragm may be delivered to the left ventricular electrodes 40 and/or 42 and right atrial electrodes 20 and/or 22 to an indifferent electrode such as the housing electrode 26 or one of the coil electrodes 34 or 36. HV rated capacitors included in defibrillation therapy module 120 may be controlled to generate the HV diaphragmatic pacing pulses delivered to selected electrodes 20, 22, 30, 32, 40 and 42 using HV switching circuitry 136. Other apparatus and techniques that may be used for activating the diaphragm in an attempt to increase cardiac output are generally disclosed in U.S. Pat. No. 7,277,757 (Casavant, et al.), hereby incorporated herein by reference in its entirety.

Controller 112 may be embodied as a processor including any one or more of a microprocessor, a digital signal processor (DSP), an application specific integrated circuit (ASIC), a field-programmable gate array (FPGA), or equivalent discrete or integrated logic circuitry. In some examples, controller 112 may include multiple components, such as any combination of one or more microprocessors, one or more controllers, one or more DSPs, one or more ASICs, or one or more FPGAs, as well as other discrete or integrated logic circuitry. The functions attributed to controller 112 herein may be embodied as software, firmware, hardware or any combination thereof. Controller 112 includes a therapy control unit that controls therapy module 104 to deliver therapies to heart 12 according to a selected one or more therapy programs, which may be stored in memory 108. Controller 112 associated memory 108 are coupled to the various components of IMD 10 via a data/address bus. Memory 108 stores intervals, counters, or other data used by controller 112 to control sensing module 102, therapy delivery module 104 and telemetry module 106. Such data may include intervals and counters used by controller 112 for detecting a heart rhythm and to control the delivery of therapeutic pulses to heart 12. Memory 108 also stores intervals for controlling cardiac sensing functions such as blanking intervals and refractory sensing intervals. Events (P-waves and R-waves) sensed by sensing module 102 may be identified based on their occurrence outside a blanking interval and inside or outside of a refractory sensing interval.

Memory 108 may store computer-readable instructions that, when executed by controller 112, cause IMD 10 to perform various functions attributed throughout this disclosure to IMD 10. The computer-readable instructions may be encoded within memory 108. Memory 108 may comprise non-transitory computer-readable storage media including any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media, with the sole exclusion being a transitory propagating signal.

Tachyarrhythmia detection algorithms may be stored in memory 108 and executed by controller 112 for detecting ventricular tachycardia (VT), ventricular fibrillation (VF) as well as discriminating such ventricular tachyarrhythmias, generally referred to herein as “shockable rhythms” from atrial or supraventricular tacharrhythmias, such as sinus tachycardia and atrial fibrillation (A FIB). Ventricular event intervals (R-R intervals) sensed from the EGM signals are commonly used for detecting cardiac rhythms. Additional information obtained such as R-wave morphology, slew rate, other event intervals (e.g., P-P intervals and P-R intervals) or other sensor signal information may be used in detecting, confirming or discriminating an arrhythmia. Reference is made to U.S. Pat. No. 5,354,316 (Keimel), U.S. Pat. No. 5,545,186 (Olson et al.) and U.S. Pat. No. 6,393,316 (Gillberg et al.) for examples of arrhythmia detection and discrimination using EGM signals, all of which patents are incorporated herein by reference in their entirety. The techniques described herein for detecting a HV short circuit condition and responding thereto may be implemented in the types of devices disclosed in the above-referenced patents.

In response to detecting a shockable rhythm, a programmed therapy is delivered by therapy delivery module 104 under the control of controller 112. A description of high-voltage output circuitry and control of high-voltage shock pulse delivery is provided in the above-incorporated '186 Olson patent. Typically, a tiered menu of arrhythmia therapies are programmed into the device ahead of time by the physician and stored in memory 108. For example, on initial detection of a ventricular tachycardia, an anti-tachycardia pacing therapy may be selected and delivered. On redetection of the ventricular tachycardia, a more aggressive anti-tachycardia pacing therapy may be scheduled. If repeated attempts at anti-tachycardia pacing therapies fail, a HV cardioversion pulse may be selected thereafter. Therapies for tachycardia termination may also vary with the rate of the detected tachycardia, with the therapies increasing in aggressiveness as the rate of the detected tachycardia increases. For example, fewer attempts at anti-tachycardia pacing may be undertaken prior to delivery of cardioversion pulses if the rate of the detected tachycardia is above a preset threshold.

In the event that ventricular fibrillation is identified, high frequency burst stimulation may be employed as the initial attempted therapy. Subsequent therapies may be delivery of HV defibrillation shock pulses, typically in excess of 5 Joules, and more typically in the range of 20 to 35 Joules. Lower energy levels may be employed for cardioversion. In the absence of a HV short circuit condition, the defibrillation pulse energy may be increased in response to failure of an initial pulse or pulses to terminate fibrillation. In response to detection of a high voltage short condition, the controller 112 will control therapy delivery module 104 to continue therapy attempts using alternate approaches while simultaneously preparing other responses such as emergency alerts to appropriate personnel as well as forceful paced activation of diaphragmatic musculature using pacing at high output (e.g. 100 V) in order to invoke phrenic nerve and/or direct diaphragmatic stimulation as described above.

IMD 10 may additionally be coupled to one or more physiological sensors. Physiological sensors may include pressure sensors, accelerometers, flow sensors, blood chemistry sensors, activity sensors or other physiological sensors known for use with implantable cardiac stimulation devices. Physiological sensors may be carried by leads extending from IMD 10 or incorporated in or on the IMD housing. Sensor signals may be used in conjunction with EGM signals for detecting and/or confirming a heart rhythm.

Telemetry module 106 is used for transmitting data accumulated by IMD 10 wirelessly to an external device (not shown), such as a programmer or home monitor. Examples of communication techniques used by IMD 10 include low frequency or radiofrequency (RF) telemetry, which may be an RF link established via Bluetooth, WiFi, or MICS. IMD 10 receives programming commands and algorithms from an external device via telemetry module 106. Telemetry module 106 may be controlled by controller 112 for delivering a patient or clinician alert or notification in response to detecting HV short circuit condition.

IMD 10 may optionally be equipped with alarm circuitry 110 for notifying the patient or other responder that a patient alert condition has been detected by IMD 10. In one embodiment, the alarm 110 may emit an audible tone or notification to alert the patient or a responder that immediate medical attention is required. For example, if a shockable rhythm is detected and a HV short circuit condition is detected, alarm 110 may be used to notify the patient, a caregiver or other responder such that emergency responders can be called. In some embodiments, alarm 110 calls an emergency number directly via a wireless communication network.

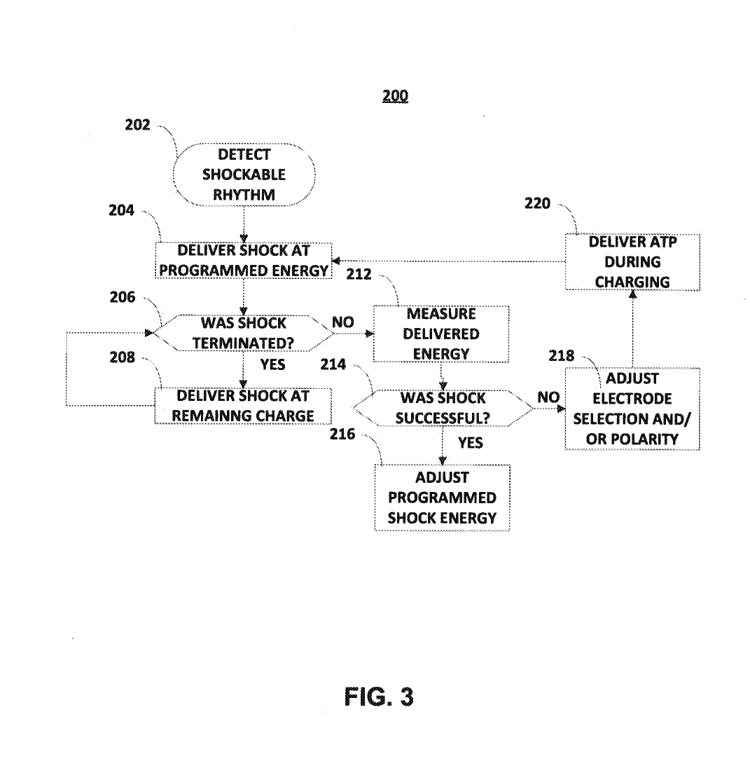

FIG. 3 is a flow chart 200 of a method for controlling delivery of a HV shock therapy to a patient. Flow chart 200 is intended to illustrate the functional operation of the IMD 10, and should not be construed as reflective of a specific form of software or hardware necessary to practice the methods described. It is believed that the particular form of software will be determined primarily by the particular system architecture employed in the IMD and by the particular detection and therapy delivery methodologies employed by the device. Providing software, hardware and/or firmware to accomplish the described functionality in the context of any modern IMD, given the disclosure herein, is within the abilities of one of skill in the art.

Methods described in conjunction with flow charts presented herein may be implemented, at least in part, in a non-transitory computer-readable medium that stores instructions for causing a programmable processor to carry out the methods described. A “non-transitory computer-readable medium” includes but is not limited to any volatile or non-volatile media, such as a RAM, ROM, CD-ROM, NVRAM, EEPROM, flash memory, or other computer-readable media, with the sole exception being a transitory, propagating signal. The instructions may be implemented as one or more software modules, which may be executed by themselves or in combination with other software by controller 112 in cooperation with therapy delivery module 104 and sensing module 102.

At block 202, a shockable rhythm is detected. As indicated above, a shockable rhythm is generally a cardiac rhythm that is treatable by delivery of a HV shock. Generally, shockable rhythms include ventricular fibrillation and may include fast ventricular tachycardia, particularly when ATP fails to terminate the VT. In response to detecting the shockable rhythm, a HV shock pulse is delivered at block 204 by the therapy delivery module 104 at a programmed shock energy. In some examples, a programmed shock energy may be 20 Joules or higher, though lower shock energies may be programmed according to patient need.

At block 206, the controller 112 determines if the HV shock was terminated by the short circuit protection circuitry 134. This determination is made by comparing the delivered energy to the programmed shock energy in one embodiment. The short circuit protection circuitry will detect a higher than expected current during the shock pulse delivery. The protection circuit will terminate pulse delivery by opening the circuit and preventing the high current from damaging IMD circuitry. The delivered energy can be computed from the known current and remaining voltage charge on the HV capacitors. For example, if a HV short circuit condition is present, short circuit protection circuit may terminate the pulse, and the controller 112 may determine that only 1 Joule was “delivered.” The HV short circuit condition, however, prevents the shock pulse from being delivered to the heart. The “delivered” energy, e.g. 1 Joule, may be compared to the programmed shock energy, e.g. 35 Joules. The large difference between the “delivered” energy and the programmed energy is detected as a terminated shock pulse due to a HV short circuit condition by controller 112 at block 206.

In response to detecting a terminated shock, the controller 112 controls therapy delivery module 104 to immediately deliver a shock using the remaining HV capacitor charge at block 208. The next charge is delivered in a “rapid fire” approach in that no adjustment to the capacitor charge is performed to either increase or decrease the capacitor voltage. In one embodiment, each time the shock is terminated due to the HV short circuit condition, the controller 112 controls therapy delivery module 104 to continue a succession of HV shock pulses delivered using the truncated charge remaining on the HV capacitors after each terminated shock. The truncated charge is the charge remaining on the HV capacitors following a terminated shock pulse and is not adjusted in any manner between pulses in the succession of HV shock pulses delivered in this rapid fire manner.

In this way, the decreasing shock energy of each successive pulse delivered using the remaining truncated capacitor charge may reach an energy that is below an arcing threshold, i.e. below a threshold that results in a HV short circuit condition. If this arcing threshold is greater than a defibrillation or cardioversion threshold, one of the shock pulses delivered in rapid succession having a pulse energy below the arcing threshold may successfully terminate the tachyarrhythmia. If a shock pulse energy is below the arcing threshold, such that a HV short circuit condition does not occur during the shock delivery, the shock pulse energy will be delivered to the heart. The shock will not be terminated prematurely by the short circuit protection circuitry at block 206. If the delivered shock is above the cardioversion/defibrillation threshold, the shock may successfully terminate the tachyarrhythmia. The rapid succession of pulses delivered using the decreasing, truncated capacitor charge after each terminated pulse enables can result in rapidly achieving a successful therapy despite the HV short circuit condition. The absence of any adjustment, i.e. increase or decrease, of the HV capacitors from a truncated charge remaining after a terminated pulse, can save time in reaching a successful shock energy.

In some embodiments, no cardiac rhythm analysis is performed between the successively delivered truncated shocks. A terminated shock is assumed to have failed in terminating the tachyarrhythmia and the successive shocks continue in a rapid fire manner unit a shock pulse is not terminated. In alternative embodiments, a confirmation of sustained tachyarrhythmia may be performed between successive shocks. For example, a brief confirmation algorithm may be performed at block 206 when a determination is made whether the shock has been terminated to also confirm if the tachyarrhythmia is sustained. A required number of tachyarrhythmia detection intervals, e.g. 3 to 5 short intervals within a detection zone, may be required between successive shocks to verify the tachyarrhythmia as being sustained prior to the next truncated shock delivery.

In response to a shock pulse delivered in rapid succession not being terminated (block 206), the delivered energy is measured at block 212. This energy is below the arcing threshold. The programmed shock energy for the current shock delivery electrode vector may be adjusted to this delivered energy to avoid a HV short circuit condition during future HV therapy at block 216. Prior to adjusting the programmed shock energy, the controller 112 may verify that the delivered shock was successful in terminating the tachyarrhythmia at block 214. If the shock was not terminated and the tachyarrhythmia was terminated, the delivered shock energy was below a HV short circuit threshold and above a defibrillation or cardioversion threshold. The programmed shock energy is appropriately reprogrammed to the delivered shock energy at block 216.

If the delivered shock was not terminated but did not successfully terminate the tachyarrhythmia, the truncated capacitor charge resulting in a shock energy below a HV short circuit threshold may also be below a defibrillation or cardioversion threshold. In this case, reprogramming the shock energy to the delivered energy may not be appropriate since the shock energy did not successfully terminate the tachyarrhythmia. In this situation, the controller 112 may select different electrodes and/or change electrode polarity assignment for delivering shock pulses in an attempt to eliminate the HV short circuit condition.

In one embodiment, if both the RV coil electrode 34 and the SVC coil electrode 36 are selected in combination with the housing electrode 26, the SVC coil electrode may be eliminated from the shock delivery vector such that the next shock is delivered using the RV coil electrode 34 and the housing electrode 26. In another embodiment, the polarities of the RV coil electrode 34 and the SVC coil electrode 36 may be reversed. Switching the electrode polarity to a reverse polarity assignment during shock delivery may prevent a HV short circuit condition.

Switching the electrode polarity within a given electrode vector selection is a different response than changing the selected electrodes to produce an electrode vector. The same electrodes and associated conductors will still be used in delivering the shock pulse, just in a reversed polarity, as opposed to eliminating an electrode and its associated conductor from a shock delivery electrode vector selection. While a conductor that may be associated with the HV short circuit condition may remain utilized in the reversed polarity configuration, this reversed polarity may result in a HV short circuit threshold that is lower than the short circuit threshold for the original polarity. Accordingly, one response to a HV short circuit condition may be to reverse the polarity of the existing electrodes being used for delivering the shock that was terminated.

Since the previous non-terminated shock was not successful in terminating the tachyarrhythmia (block 214), after adjusting the electrode selection or reversing a polarity of the selected electrodes at block 218, a new shock is delivered at block 204. It is contemplated that during charging of the capacitors after unsuccessfully terminating the detected tachyarrhythmia, ATP may be delivered at block 220. Prior to delivering the shock at block 204, the controller 112 may confirm that the shockable tachyarrhythmia is still being detected.

It is contemplated that at any point in responding to a HV short circuit condition, controller 112 may control therapy delivery module 104 to deliver ATP therapy in an attempt to terminate the tachyarrhythmia. For example, ATP may be delivered for short periods between shock attempts delivered at block 208 using successively truncated capacitor charge, during recharging of capacitors at block 220, or after all shock electrode configurations and/or polarities have been attempted without success. In some cases, ATP may successfully terminate a tachyarrhythmia even when a delivered shock has been unsuccessful.

The use of ATP delivered at any of time point after termination of shock pulse may include verification of stable RR intervals by controller 112. For example, at block 220, before starting ATP, RR interval stability may be verified by measuring a predetermined number of RR intervals and determining an RR interval range less than a predetermined threshold, e.g. less than approximately 30 ms, in one embodiment. At any point that ATP is delivered, the controller 112 may reconfirm tachyarrhythmia detection before delivering a next shock.

The process shown in flow chart 200 may be repeated if a shock delivered at block 204 using an alternate electrode vector or polarity is also terminated by the HV short circuit protection circuitry. Initially, a rapid succession of shock pulses are delivered without changing electrode selection or polarity using the remaining truncated capacitor voltage in an attempt to deliver a shock below the high voltage short circuit threshold but above a cardioversion or defibrillation threshold. If a shock is successful in terminating the rhythm, the currently selected electrode vector and polarity will remain programmed as the shock delivery electrode selection and the successful shock energy will be programmed as the shock pulse energy to be used in future shock therapies. If the adjusted electrode selection or reversed polarity fails to terminate the rhythm, the controller 112 may continue to select different electrode vectors and/or polarity assignments from the available electrodes until a delivered shock is successful in terminating the rhythm. The controller 112 may store in memory 108 which electrode combinations and polarities result in a terminated shock and eliminate such electrode configurations and/or polarities from future shock attempts.

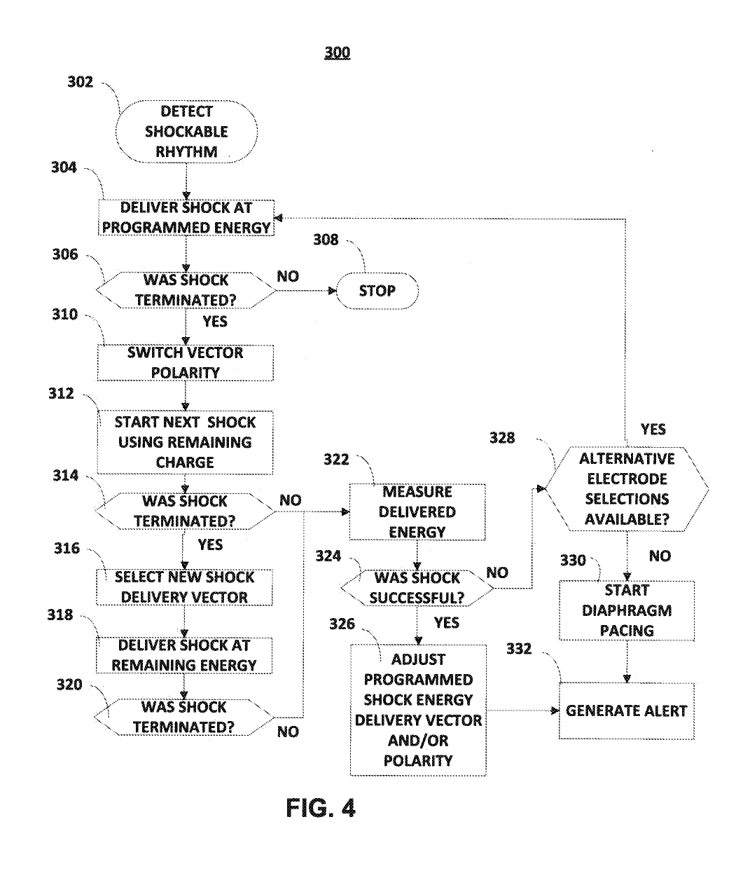

FIG. 4 is a flow chart 300 of a method for controlling electrical stimulation therapy in response to detecting a shockable rhythm according to an alternative embodiment. At block 302, a shockable rhythm, e.g. a VT or VF is detected. A shock therapy is delivered at block 304 according to a programmed pulse energy. The controller 112 determines if the shock was terminated prior to completion of capacitor discharge at block 306 by the HV short circuit protection circuit.

As described previously, determination of whether a shock has been terminated prior to complete capacitor discharge may include comparing a delivered energy to the programmed pulse energy. The delivered energy can be estimated or computed by measuring the voltage on the high voltage capacitor(s) prior to delivery (upon charge completion) and after termination of the shock pulse. The difference between the capacitor voltage before delivery and after termination is directly correlated to the energy delivered (less resistive losses) and may be used as an estimate of the delivered energy. Alternatively, the delivered energy may be estimated by computation (e.g. Edelivered=½ C [V₁²−V₂²] where C is the capacitance and V1 and V2 are the capacitor voltages measured before and after shock delivery respectively). In another example, the determination of whether a shock has been terminated by the HV short circuit protection circuit may include analyzing a measured impedance during shock delivery.

If the controller 112 determines that the shock was terminated prematurely, the shock vector polarity is reversed at block 310. A shock is immediately delivered using the remaining truncated capacitor charge, absent any recharging, discharging or otherwise adjusting the truncated capacitor charge remaining at the time the previous charge was terminated. If the shock is started but terminated by the HV short circuit protection circuitry, as determined at block 314, the controller may continue a rapid succession of shock pulses, each pulse started using a truncated capacitor charge remaining upon terminating the preceding pulse, until a shock is delivered without being terminated by the HV short circuit protection circuitry. Alternatively, after a predetermined number of shock attempts using the reversed polarity, e.g. one or more shock attempts, a different shock vector may be selected at block 316. Selecting a different shock vector involves eliminating or replacing an electrode from the electrode vector used to deliver the shock that was terminated at block 314, e.g. eliminating the SVC coil electrode 36. A rapid succession of shocks are attempted using the truncated, remaining capacitor charge after each terminated pulse until a shock is not terminated by the HV short circuit protection circuitry.

In some embodiments, selection of a different shock vector (i.e. electrode combination) or switching a polarity of a selected shock vector may be changed on each successive shock delivery or after another predetermined number of the successive shock attempts. For example, two shocks may be attempted in rapid succession using the original electrode selection and polarity, two shocks may be attempted in rapid succession using a reversed polarity of the original electrode selection, two shocks may be attempted using a different electrode selection, two shocks using a reversed polarity of the different electrode selection and so on until a shock is delivered that is not terminated. It is recognized that numerous variations can be conceived for switching electrode vector selection and/or electrode polarity between successive shocks. Each successive shock, however, is delivered using a truncated charge remaining on the HV capacitors upon termination of a preceding shock, regardless of a change in electrode polarity or electrode selection, and lacking any adjustment of the remaining capacitor charge.

In some embodiments, selection of a new shock delivery vector at block 316 may include selecting pacing or sensing electrodes that are normally used for delivering LV pacing pulses or sensing EGM signals. For example, with reference to the lead and electrode configuration shown in FIG. 1, the RV tip and ring electrodes 30 and 32 may be selected with a common polarity to function as a combined “high voltage” electrode for delivering a shock. The RV tip and ring electrodes 30 and 32 may be electrically coupled together with the CS lead tip and ring electrodes 40 and 42 and/or the RA lead tip and ring electrodes 20 and 22 to form a multi-point HV electrode combination for delivering a shock pulse. The pacing/sensing electrodes 20, 22, 30, 32, 40 and 42 may be electrically coupled together in any combination and selected with the housing electrode 26 or a coil electrode 34 or 36 for delivering a shock pulse.

In one example, if a shock is terminated at block 306 when the RV coil 34 and SVC coil 36 are selected in combination with the IMD housing electrode 26, an initial attempt of reversing a polarity of the selected electrodes may be made. If the shock is again terminated, the SVC coil 36 may be eliminated from the selected shock vector and an attempt may be made to deliver the next shock at block 316 between the RV coil 34 and the housing electrode 26. If this shock is also terminated, suggesting a short circuit condition involving the conductor extending to the RV coil electrode 34, the controller 112 may switch the electrode selection to include multiple pace/sense electrodes electrically tied together to form a multi-point pole for the next attempted shock delivery.

If a shock is not terminated (at block 314 or block 320) the delivered energy is measured at block 322. If the shock was successful in terminating the tachyarrhythmia, the adjusted electrode selection and/or polarity and associated successful shock energy are stored for use in delivering future shock therapies at block 326.

An alert may be generated at block 332 to notify the patient or a clinician that a HV short circuit condition has caused the shock energy and/or shock delivery electrode selection to be reprogrammed. The alert enables a clinician to take corrective action to replace a lead if necessary to eliminate future HV short circuit conditions. The alert in this situation may be a telecommunication transmission, e.g. to a smart phone or a 911 call, using BLUETOOTH® open wireless technology or other wireless telecommunication system, such that a clinician or other medical responder can act promptly in addressing the high voltage fault situation.

If a non-terminated shock fails to terminate the tachyarrhythmia, as determined at block 324, the controller 112 determines if additional electrode selections may be made at block 328. All available electrode combinations and/or polarities may not be tested before the remaining capacitor charge falls below a defibrillation/cardioversion threshold, or, by the time a particular electrode vector is selected, the remaining charge may result in a shock below the defibrillation/cardioversion threshold. Accordingly, the controller 112 may reselect an electrode combination or polarity configuration to be retested starting from a full capacitor charge by returning to block 304 or select a new combination or polarity that has not yet been tested.

If all available electrode selections and polarities have been tested or a maximum number of attempts has been reached, the controller 112 may initiate diaphragm pacing at block 330. Diaphragm pacing may be performed through direct diaphragm pacing or through stimulation of the right and/or left phrenic nerves as described above. Capture of the diaphragm or phrenic nerves may be achieved by selecting pace/sense electrodes 20, 22, 30, 32, 40 and/or 42 and delivering relatively high amplitude pacing pulses to promote a high likelihood of diaphragm activation and cyclic thoracic cavity pressure changes. Methods for respiratory nerve stimulation generally disclosed in the above incorporated U.S. Pat. No. 7,277,757 (Casavant, et al.) may be adapted for use in delivering diaphragm pacing at block 330.

By pacing the diaphragm, an increase in cardiac output may be achieved which may be enough to sustain a patient until emergency responders arrive. Upon initiating diaphragm pacing at block 330, an alert may be generated at block 332 to notify a caregiver or other nearby responder that urgent medical attention is needed, enabling an emergency 911 call to be made, for example. Alternatively, the controller may cause the telemetry module 110 to make a 911 call or other communication directly to an emergency responder.

The various responses described in conjunction with the flow charts 200 and 300 presented herein may be performed in any combination and may be performed in a different order than the illustrative examples provided. In various embodiments, responses to termination of a shock pulse due to a HV short circuit condition may be added or removed depending on the particular system in which the described techniques are being implemented and the conditions under which a shock is terminated, such as the detected rhythm, the remaining IMD battery charge, the available electrode configuration or other conditions.

Thus, a medical device and associated method for controlling delivery of a high voltage therapy have been presented in the foregoing description with reference to specific embodiments. It is appreciated that various modifications to the referenced embodiments may be made without departing from the scope of the disclosure as set forth in the following claims.

1. An implantable medical device, comprising:
a therapy delivery module comprising a high voltage therapy delivery circuit;
a high voltage short circuit protection circuit configured to terminate delivery of a high voltage pulse by the therapy delivery module in response to a short circuit condition;
a sensing module for detecting a need for a high voltage therapy; and
a therapy control unit configured to control the therapy delivery module to deliver a shock pulse in response to detecting the need for the high voltage therapy, detect a termination of the high voltage pulse, a truncated shock charge remaining on the high voltage therapy delivery circuit upon terminating the high voltage pulse, the therapy control unit configured to control the therapy delivery module to deliver a next shock pulse at the remaining truncated shock charge.

2. The device of claim 1, wherein the therapy control unit is configured to control the therapy delivery module to repeatedly deliver shock pulses at sequentially truncated shock charges in response to repeated termination of the shock pulses by the high voltage short circuit protection circuit.

3. The device of claim 1, further comprising a plurality of high voltage electrodes coupled to the therapy delivery module,
the therapy control unit configured to deliver the shock pulse using a first high voltage electrode configuration and the truncated shock pulse using a second high voltage electrode configuration different than the first high voltage electrode configuration.

4. The device of claim 3, wherein the first high voltage electrode configuration comprises a first electrode having a first polarity and a second electrode having a second polarity and the second high voltage electrode configuration comprises the first electrode having the second polarity and the second electrode having the first polarity.

5. The device of claim 1, further comprising:
a plurality of high voltage electrodes coupled to the therapy delivery module;
a plurality of low voltage electrodes coupled to the therapy delivery module;
the therapy control module configured to select a first electrode configuration comprising the high voltage electrodes for delivering the shock pulse and to select a second electrode configuration comprising the plurality of low voltage electrodes for delivering the next shock pulse.

6. The device of claim 1, wherein the therapy control module detects the termination of the shock pulse by determining a delivered energy is less than a programmed energy.

7. The device of claim 1, wherein the therapy control module detects the termination of the shock pulse by detecting a low impedance during the shock delivery.

8. The device of claim 1, further comprising:
a plurality of electrodes coupled to the therapy delivery module,
the therapy control module being configured to control the therapy delivery module to deliver pacing pulses to selected ones of the plurality of electrodes for pacing a diaphragm of the patient subsequent to termination of the shock pulse.

9. The device of claim 8, wherein the therapy control module controls the therapy delivery module to deliver charge stored by the high voltage therapy delivery circuitry via the selected ones of the plurality of electrodes for pacing the diaphragm.

10. The device of claim 1, wherein the therapy control unit is configured to control the therapy delivery module to deliver anti-tachycardia pacing therapy after delivering the truncated shock pulse.

11. The device of claim 1, wherein the therapy control module stores an electrode vector configuration associated with a terminated shock pulse.

12. A method, comprising:
controlling a therapy delivery module comprising a high voltage therapy delivery circuit to deliver a shock pulse in response to a sensing module detecting a need for a high voltage therapy;
enabling a therapy control unit to detect a termination of the shock pulse by a high voltage shock protection circuit, a truncated shock charge remaining on the high voltage therapy delivery circuit upon terminating the shock pulse; and
controlling the therapy delivery module to deliver a next shock pulse at the remaining truncated shock charge.

13. The method of claim 12, further comprising enabling the therapy control unit to control the therapy delivery module to repeatedly deliver shock pulses at sequentially truncated shock charges in response to repeated termination of the shock pulses by the high voltage short circuit protection circuit.

14. The method of claim 12, further comprising controlling the therapy delivery module to deliver the shock pulse using a first high voltage electrode configuration and the truncated shock pulse using a second high voltage electrode configuration different than the first high voltage electrode configuration.

15. The method of claim 14, further comprising selecting the first high voltage electrode configuration to comprise a first electrode having a first polarity and a second electrode having a second polarity and selecting the second high voltage electrode configuration to comprise the first electrode having the second polarity and the second electrode having the first polarity.

16. The method of claim 12, further comprising:
selecting a first electrode configuration comprising a plurality of high voltage electrodes for delivering the shock pulse; and
selecting a second electrode configuration comprising a plurality of low voltage electrodes for delivering the next shock pulse.

17. The method of claim 12, further comprising enabling the therapy control module to detect the termination of the shock pulse by determining a delivered energy is less than a programmed energy.

18. The method of claim 12, further comprising enabling the therapy control module to detect the termination of the shock pulse by detecting a low impedance during the shock delivery.

19. The method of claim 12, further comprising:
enabling the therapy control unit to control the therapy delivery module to deliver pacing pulses to selected ones of a plurality of electrodes for pacing a diaphragm of the patient subsequent to termination of the shock pulse.

20. The method of claim 19, further comprising delivering charge stored by the high voltage therapy delivery circuitry via the selected ones of the plurality of electrodes for pacing the diaphragm.

21. The method of claim 12, further comprising delivering anti-tachycardia pacing therapy after delivering the truncated shock pulse.

22. The method of claim 12, further comprising storing an electrode vector configuration and polarity associated with a terminated shock pulse.

23. A non-transitory, computer-readable medium storing a set of instructions which cause a control unit of an implantable medical device to perform a method, the method comprising:
controlling a therapy delivery module comprising a high voltage therapy delivery circuit to deliver a shock pulse in response to a sensing module detecting a need for a high voltage therapy;
enabling a therapy control unit to detect a termination of the shock pulse by a high voltage shock protection circuit, a truncated shock charge remaining on the high voltage therapy delivery circuit upon terminating the shock pulse; and
controlling the therapy delivery module to deliver a next shock pulse at the remaining truncated shock charge.
